(19) United States
(12) Patent Application Publication
(10) Pub. No.: US 2001/0007083 A1
(43) Pub. Date: Jul. 5, 2001

(54) Device and active component for inhibiting formation of thrombus-inflammatory cell matrix

(76) Inventor: Wouter E. Roorda, Palo Alto, CA (US)

Correspondence Address: Squire, Sanders & Dempsey L.L.P., Suite 300, One Maritime Plaza, San Francisco, CA, 94111, US

(21) Appl. No.: 09/748,412

(22) Filed: Dec. 21, 2000

Related U.S. Application Data
(63) Continuation-in-part of application No. 09/475,957, filed on Dec. 29, 1999.

(51) Int. Cl. A61F 2/06

(52) U.S. Cl. 623/1.15; 623/1.43

(57) ABSTRACT

A combination drug treatment for inhibiting stenosis or restenosis is disclosed. The combination treatment is an active component containing both an anti-inflammatory substance and an anti-thrombotic substance which, together, contribute to an inhibiting effect on the initial stages of stenosis or restenosis. The active component can be delivered to a site of treatment by being carried on a device, such as a stent.

### CROSS-REFERENCE

This is a continuation-in-part of U.S. patent application Ser. No. 09/475,957, filed on Dec. 29, 1999.

### BACKGROUND OF THE INVENTION

1. Field of the Invention

The present invention relates generally to an active composition for inhibiting restenosis. In one embodiment, the invention relates generally to use of the active composition in conjunction with a vascular device or a polymeric matrix so that the composition is delivered and applied to the treatment site.

2. Description of the Related Art

Percutaneous transluminal coronary angioplasty (PTCA) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across an occlusive lesion. Once in position across the lesion, the balloon is inflated to a predetermined size to radially press against the atherosclerotic plaque of the lesion for remodeling the vessel wall. The balloon is then deflated to a smaller profile to allow the catheter to be withdrawn from the patient's vasculature.

A complication associated with the above procedure is that reocclusion of the artery due to aggressive scar tissue growth, a process known as restenosis, may develop over several months after the procedure. Restenosis is thought to involve the body's natural healing process. Angioplasty or other vascular surgeries injure the arterial wall, removing the vascular endothelium, disturbing the underlying intima and causing death of medial smooth muscle cells. Excessive neointimal tissue formation, characterized by smooth muscle cell migration and proliferation into the intima, follows the injury. The extensive thickening of this tissue narrows the lumen of the blood vessel, constricting or blocking blood flow through the artery.

To reduce the chance of developing restenosis, an expandable intraluminal prosthesis, one example of which includes a stent, is implanted in the lumen of the artery to maintain vascular patency. Stents are scaffoldings, usually cylindrical or tubular in shape, which function to physically hold open and, if desired, to expand the wall of a passageway. Typically stents are compressible, so that they can be inserted through small cavities via small catheters, and then expanded to a larger diameter once they are delivered to a desired location. Stents are also capable of securing therapeutic substances and locally releasing such substances for a predetermined duration of time. This allows high concentrations of therapeutic substances to be delivered directly to a treatment site. Examples in patent literature disclosing stents which have been successfully applied in PTCA procedures include stents illustrated in U.S. Pat. No. 4,733,665 issued to Palmaz, U.S. Pat. No. 4,800,882 issued to Gianturco, and U.S. Pat. No. 4,886,062 issued to Wiktor.

Restenosis frequently occurs at the site of stent implantation, reducing the effectiveness of stent therapy. When restenosis does occur in the stented segment, its treatment can be challenging, as clinical options are more limited as compared to lesions that were treated solely with a balloon. A method for inhibiting restenosis at a stent implantation site would reduce the mortality rate associated with restenosis.

To inhibit restenosis, therapeutic agents hoped to counter important steps in the formation of the neointimal tissue, particularly the migration and proliferation of smooth muscle cells, are being developed. For example, on the discovery that platelet derived growth factor (PDGF) stimulates smooth muscle cell growth at arterial lesions, the administration of monoclonal anti-PDGF receptor antibodies is being advanced. Similarly, secretory T lymphocyte protein interferon-gamma, which has also been shown to inhibit smooth muscle growth, is being tested, but so far is unable to adequately inhibit restenosis. Additional pharmacological therapies, such as the administration of heparin to inhibit thrombus formation, calcium channel blockers to reduce platelet aggregation, and angiotensin agonists to prevent vasoconstriction have also met with limited success.

Therefore, there is a need to sufficiently inhibit restenosis at a stent site, to greatly improve the effectiveness of coronary stents, and to improve the effectiveness of any long-term or permanent devices implanted within a blood vessel. There is also a need for a better active composition to inhibit restenosis.

### SUMMARY OF THE INVENTION

In accordance with one aspect a method for inhibiting restenosis of a blood vessel, e.g., a coronary artery, a peripheral vessel, and alike, is provided. The method includes providing a device carrying an active component—the active component comprises at least one anti-thrombotic substance in combination with at least one anti-inflammatory substance; and implanting the device into the blood vessel to inhibit restensosis of the blood vessel. The device can be a balloon-expandable stent, a self-expandable stent, or a graft. In one embodiment, the device can be coated with an ethylene vinyl alcohol copolymer, the active component being contained in the ethylene vinyl alcohol copolymer.

Representative examples of the anti-thrombotic substance include heparin, sodium heparin, low molecular weight heparin, hirudin, argatroban, forskolin, vapiprost, prostacyclin and prostacyclin analogs, D-phe-pro-arg-chloromethylketone, dipyridamole, glycoprotein IIb/IIIa platelet membrane receptor antibody, and recombinant hirudin. Representative examples of the anti-inflamatory substance include aspirin, diclofenac, etodolac, ibuprofen, ketoprofen, ketorolac, nabumetone, naproxen, oxaprozin, clobetasol, diflucortolone, flucinolone, halcinolonide, halobetasol, dexamethasone, betamethasone, corticol, cortisone, prednisone, and prednisolone.

In accordance with another aspect of the invention, a stent is provided for implantation in a mammalian blood vessel. The stent can be coated with an anti-thrombogenic material which is not substantially released from the stent when the stent is implanted in the blood vessel. An anti-inflammatory substance is contained in the coating and capable of being released from the coating when the stent is implanted. In one embodiment the coating is made from a hydro-gel, such as poly-ethylene oxide, albumin, hydrophilic poly-methacrylates and hydrophilic poly urethanes.

In accordance with another embodiment a stent is provided having pores formed in the surface. The stent is made from an anti-thrombogenic material and the pores can contain an anti-inflammatory substance.

In accordance with another aspect of the invention, a polymeric matrix comprising an active component for inhibiting the migration or proliferation of smooth cells is provided. The active component inhibits the formation of thrombus and inhibits the infiltration of inflammatory cells in the thrombus. The polymeric matrix can be a liposome or an ethylene vinyl alcohol copolymer.

### DETAILED DESCRIPTION

It is believed that the etiology of restenosis following stent implantation includes thrombus accumulation, in which clots of blood having a high concentration of platelets attach to the stent struts. Inflammatory cells, mainly macrophages, then infiltrate the thrombus in large numbers, to develop a thrombus-inflammatory cell matrix. Platelets and macrophages in the thrombus-inflammatory cell matrix secrete chemical messengers such as cytokines and growth factors that cause smooth muscle cells to migrate and proliferate at the stent site. A distinct layer of neointimal tissue forms as the smooth muscle cells continue to proliferate and aggregate at the stent site, eventually causing occlusion of the lumen of the blood vessel. Accordingly, a device and an active component for inhibiting the formation of the thrombus-inflammatory cell matrix to inhibit the activity of vascular smooth muscle cells are provided. More specifically, the activity of smooth muscle cells which is inhibited includes abnormal or inappropriate migration and/or proliferation of smooth muscle cells.

“Thrombus” is an aggregation of blood factors, primarily platelets and fibrin with entrapment of cellular elements and/or red blood cells.

“Platelets” are particles found in the bloodstream that bind to fibrinogen at the site of a wound to begin the blood clotting process.

“Fibrin” is an insoluble protein formed from fibrinogen by the proteolytic action of thrombin during normal clotting of blood.

“Macrophage” is a relatively long-lived phagocytic cell of mammalian tissue, derived from blood monocyte.

“Smooth muscle cells” include those cells derived from the medial and adventitia layers of the vessel which proliferate in intimal hyperplastic vascular sites following vascular trauma or injury. Under light microscopic examination, characteristics of smooth muscle cells include a histological morphology of a spindle shape with an oblong nucleus located centrally in the cell with nucleoli present and myofibrils in the sarcoplasm. Under electron microscopic examination, smooth muscle cells have long slender mitochondria in the juxtanuclear sarcoplasm, a few tubular elements of granular endoplasmic reticulum, and numerous clusters of free ribosomes. A small Golgi complex may also be located near one pole of the nucleus.

“Migration” of smooth muscle cells means movement of these cells in vivo from the medial layers of a vessel into the intima, such as may also be studied in vitro by following the motion of a cell from one location to another, e.g., using time-lapse cinematography or a video recorder and manual counting of smooth muscle cell migration out of a defined area in the tissue culture over time.

“Proliferation” of smooth muscle cells means increase in cell number.

“Abnormal” or “inappropriate” proliferation means division, growth and/or migration of cells occurring more rapidly or to a significantly greater extent than typically occurs in a normally functioning cell of the same type, i.e., hyper-proliferation.

“Inhibiting” cellular activity means reducing, delaying or eliminating smooth muscle cell hyperplasia, restenosis, and vascular occlusions, particularly following biologically or mechanically mediated vascular injury or trauma or under conditions that would predispose a mammal to suffer such a vascular injury or trauma. As used herein, the term “reducing” cellular activity means decreasing the intimal thickening that results from stimulation of smooth muscle cell proliferation. “Delaying” cellular activity means retarding the progression of the hyper-proliferative vascular disease or delaying the time until onset of visible intimal hyperplasia, as observed, for example, by histological or angiographic examination. “Elimination” of restenosis following vascular trauma or injury means completely “reducing” and/or completely “delaying” intimal hyperplasia in a patient to an extent which makes it no longer necessary to surgically intervene, i.e., to re-establish a suitable blood flow through the vessel by, for example, repeat angioplasty, atheroectomy, or coronary artery bypass surgery. The effects of reducing, delaying, or eliminating restenosis may be determined by methods known to one of ordinary skill in the art, including, but not limited to, angiography, ultrasonic evaluation, fluoroscopy imaging, fiber optic visualization, or biopsy and histology. Biologically mediated vascular injury includes, but is not limited to injury caused by or attributed to autoimmune disorders, alloimmune related disorders, infectious disorders including endotoxins and herpes viruses such as cytomegalovirus, metabolic disorders such as atherosclerosis, and vascular injury resulting from hypothermia and irradiation. Mechanical mediated vascular injury includes, but is not limited to vascular injury caused by catheterization procedures or vascular scraping procedures such as stent therapy, percutaneous transluminal coronary angioplasty, vascular surgery, transplantation surgery, laser treatment, and other invasive procedures which disrupted the integrity of the vascular intima or endothelium. The active component of the invention is not restricted in use for therapy following vascular injury or trauma; rather, the usefulness of the component will also be determined by the component's ability to inhibit cellular activity of smooth muscle cells or to inhibit the development of restenosis.

The dosage or concentration of the active component required to produce a favorable therapeutic effect should be less than the level at which the active component produces toxic effects and greater than the level at which non-therapeutic results are obtained. The dosage or concentration of the active component required to inhibit the desired activity of the vascular region can depend upon factors such as the particular circumstances of the patient; the nature of the trauma; the method of administration; the time over which the active component administered resides at the vascular site; and the nature and type of the substance or combination of substances. Therapeutic effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

The active component includes one or more anti-inflammatory substances used in combination with one or more anti-thrombotic substances, so that the active component delivers both an anti-inflammatory and an anti-thrombotic effect, disrupting the organization process of the thrombus-inflammatory cell matrix.

Anti-thrombotic substances are substances that contribute to the effect of preventing the accumulation of thrombus and include, but are not limited to, thrombin inhibitors and platelet inhibitors. Representative examples of anti-thrombotic substances include, but are not limited to, heparin, heparin derivatives, sodium heparin, low molecular weight heparin, hirudin, argatroban, forskolin, vapiprost, prostacyclin and prostacyclin analoges, D-phe-pro-arg-chloromethylketone (synthetic antithrombin), dipyridamole, glycoprotein IIb/IIIa platelet membrane receptor antibody, recombinant hirudin, thrombin inhibitor (available from Biogen), and 7E-3B® (an antiplatelet drug from Centocore).

Anti-inflammatory substances from both the non-steroidal anti-inflammatory (NSAIDS) and steroidal class may be used either alone or in combination. Examples of NAIDS include, but are not limited to, aspirin, diclofenac, etodolac, ibuprofen, ketoprofen, ketorolac, nabumetone, naproxen, and oxaprozin. Examples of steroidal anti-inflammatories include, but are not limited to, clobetasol, diflucortolone, flucinolone, halcinolonide, halobetasol, dexamethasone, betamethasone, corticol, cortisone, prednisone, and prednisolone.

The potency and half-life in situ of the therapeutic substances chosen for the active component will affect formula parameters, such as the ratio of anti-inflammatory substance to anti-thrombotic substance, and release profile parameters, such as the rate and duration of release and the cumulative amount of substance released. Determination of these specific parameters based on the substances chosen is understood by one of ordinary skill in the art.

In one embodiment, a device, one example of which includes a stent, carries the active component. Upon implantation of the device in a patient's body, the active component is locally released into the blood vessel for a duration of time. Release of the active component can usefully start immediately from the time of implantation. As a general rule, but not strictly bound by this proposition, the longer the duration of release the more effective the cocktail of anti-inflammatory substance and anti-thrombotic substance will be in inhibiting restenosis. In one embodiment, the cocktail can be released over a one week period. For an effective treatment, both sub-components of the active component can be released at the same time, because blocking initial formation of the thrombus-inflammatory cell matrix can be achieved by the presence of both substances. In another embodiment, the sub-components of the active component may be released at different times.

Methods for applying the active component to a stent include, but are not limited to, coating the device with a bio-soluble, bio-degradable, and/or bio-stable polymeric material and impregnating the material with the active component; constructing the device of porous material and securing the active component directly into the pores of the device; or incorporating the active component into a polymeric sheath that encompasses the device. Examples in the patent literature of methods of preparing medicated stent devices include U.S. Pat. No. 5,383,928 issued to Scott et al.; U.S. Pat. No. 5,980,972 to Ding; U.S. Pat. No. 5,843,172 to Yan; and U.S. Pat. No. 5,951,586 issued to Berg et al.

The desired release profile, which includes parameters such as the rate and duration of release, and the cumulative amount of substance released, may be determined, as described above, based on the characteristics of the substances chosen for the active component. Implementation of the desired release profile can be achieved by varying device design factors in consideration of the solubility in situ of the substances. By way of example only, if a therapeutic substance is highly water soluble, the release rate of the substance can be slowed down by converting the substance into a salt form with lower water solubility. Alternatively, the release rate of a highly water soluble substance may be slowed down by choosing a derivative or analog substance with a lower water solubility. The release rate of the substance can also be controlled by varying its solubility in the polymer coating. In general, the lower the solubility of the substance in a polymeric coating, the slower its release rate. Therefore, after an appropriate substance has been chosen, a polymeric coating can be selected in which the substance has the appropriate solubility. The release profile can also be adjusted, for example, by varying the number and thickness of polymer layers, with or without the active compoenent. The interrelation and correlation of these and other design factors for achieving a desired release profile of the therapeutic substances are understood by one of ordinary skill in the art.

Representative example of bio-soluble or big-degradable polymeric materials include, but are not limited to, polycaprolactone (PCL), poly-DL-lactic acid (DL-PLA), poly-L-lactic acid (L-PLA), polyorthoesters, polyiminocarbonates, aliphatic polycarbonates, and polyphosphazenes. Bio-soluble or big-degradable materials are capable of being broken down and gradually absorbed or eliminated by the body. Release of the active component occurs as these polymers dissolve or degrade in situ. Representative examples of bio-stable polymeric materials include, but are not limited to, polymers of polyurethanes, polyethylenes, polyethylene teraphthalates, ethylene vinyl acetates, silicones and polyethylene oxide. Ethylene vinyl alcohol copolymers also function effectively. Biostable polymers may be permeable to the active component, which is released by diffusion through and out of the polymeric coating.

In another embodiment, instead of the anti-thrombotic substance of the active component being released, the device, e.g., stent, is coated with an anti-thrombogenic material which is not substantially released from the device. In this embodiment, the anti-inflammatory substance is releasably contained in the anti-thrombogenic coating. Together, the anti-thrombogenic coating and the releasably contained anti-inflammatory substance achieve the effect of inhibiting the development of restenosis by deterring the formation of the thrombus-inflammatory cell matrix at the device. Release of the anti-inflammatory substance can usefully start immediately from the time of implantation.

Anti-thrombogenic coatings can be made from either an active thrombin inhibitor, typically heparin, a heparin derivative, or a heparin analog, or can be made from a passively thromboresistant material, such as a hydro-gel, or any combination of active and passive thromboresistant material. A hydro-gel makes the surface of the device “slippery” to the plasma proteins involved in thrombosis, preventing the proteins from being significantly adsorbed onto the device surface. Examples of useful hydro-gels include poly-ethylene oxide, albumin, hydrophilic poly-(meth)acrylates, and hydrophilic poly-urethanes. In another embodiment, an anti-thrombotic substance may also be releasably contained with the anti-inflammatory substance in the anti-thrombogenic coating.

In yet another embodiment, the device can be fully constructed from an antithrombogenic material that is resistant to thrombus formation and is porous, so that the anti-inflammatory substance may be releasably contained in the device.

The device used in conjunction with any of the above-described embodiments may be any suitable device, for instance a prosthetic device. Examples of prosthetic devices include, but are not limited to, self-expandable stents, balloon-expandable stents, stent-grafts, and grafts. The underlying structure of the device may be any desired design. The device can be made of a metallic material, such as an alloy, or from a polymeric material. The device need not be a prosthetic device, and may be any device capable of being introduced or implanted in or about the vasculature.

In accordance with another embodiment, the active component is delivered to the treatment site via a bio-soluble or bio-degradable particles. The active component can typically be carried by the particles by being dispersed throughout, being contained within, being coated on the particles, or combinations and variations thereof. Examples in the patent literature of particles used for local drug delivery include U.S. Pat. No. 5,869,103, issued to Yeh et al.; U.S. Pat. No. 5,817,343, issued to Burke; and U.S. Pat. No. 5,171,217, issued to March et al. The particles can be delivered to the treatment site by any suitable means. Typically, the particles are delivered by injection via a delivery catheter, but any conventional delivery system or method may be used.

The desired release profile for the active component from the particles may be determined, as described above, based on the characteristics of the therapeutic substances chosen for the active component. Implementation of the release profile parameters can be achieved in the particles by choice of material used to make the particles. For instance, the release rate of the therapeutic substance can be affected by the rate at which the polymeric material bio-degrades or dissolves in situ. Also, for example, the diffusion rate of the therapeutic substances through and out of the particles will affect the release rate of the substances from the particles.

The particles are typically polymeric micro-particles or liposomes. Particles are typically constructed from materials which include, but are not limited to, synthetic polymers, natural polymers, proteins, lipids, surfactants, or carbohydrates. Polymeric particles may have a dimension of 500 μm (micron) or less, or alternatively a dimension of 50 μm or less. Dimension of between 5 and 25 μm is also functionally suitable. Representative examples of bio-degradable polymers that can be used to form the particles include, but are not limited to, polyesters; ethylene vinyl alcohol copolymer; polyglycolides; copolymers of lactide and glycolide; polyhydroxybutyrate; polycaprolactone; copolymers of lactic acid and lactone; copolymers of lactic acid and poly(ethylene glycol); copolymers of α-hydroxy acids and α-amino acids; polyanhydrides; polyorthoesters; polyphosphazenes; copolymers of hydroxybutyrate and hydroxyvalerate; poly(ethylene carbonate); copoly(ethylene carbonate); polyethylene terephthalate; or mixtures and combinations thereof. Liposomes can have a dimension of 1 μm or less, typically having a dimension of about 50 to about 800 nm (nanometers). Liposomes are typically formed from ionic and non-ionic polar lipids.

In an alternate embodiment, the particles carrying the active component may be additionally coated with a substance to alter or affect the course of the particles in situ. The particles may be coated with one or more substances that facilitate targeting of the particles to particular cells or tissues, or that inhibit undesirable endocytosis or destruction of the particles by cellular mechanisms. Usefully, the particles may be coated with a polysaccharide that inhibits the particles' uptake by macrophage cells. Since the particles are likely to be encountered by the macrophage cells, coating the particles with a polysaccharide that inhibits the particles' uptake and destruction by macrophage cells will extend the particles' half-life in situ. Coating particles is described in U.S. Pat. No. 5,981,719, issued to Woiszwillo et al.

In another embodiment, the active component is delivered by a mixture of particles. A percentage of the particles in the mixture carry the anti-thrombotic substance, and the remainder of the particles in the mixture carry an anti-inflammatory substance effective in disrupting macrophage cells. The remainder particles may be coated with a polysaccharide which promotes these particles' uptake by macrophage cells, thus specifically targeting the macrophage cells of a thrombus-inflammatory cell matrix.

Yet in another embodiment, a delivery system is provided in which a polymer that contains the active component is injected into the lesion in liquid form. The polymer can then be cured to form the implant in situ. In situ polymerization can be accomplished by photocuring or chemical reaction. Photocuring is conducted by mixing a polymer such as, but not limited to, acrylate or diacrylate modified polyethylene glycol (PEG), pluronic, polybutylene teraphthalate-co-polyethylene oxide, polyvinyl alcohol, hydroxy ethyl methacrylate (HEMA), hydroxy ethyl methacrylate-co-polyvinyl pyrrolidone, HEMA-co-PEG, or glycidol acrylate modified heparin or sulfated dextran with the active component, with or without a photosensitizer (e.g., benzophenone) or a photoinitiator (e.g., 2,2 dimethoxy 2-phenyl acetophenone, and eosin-Y). The precursor system can be activated by a suitable wavelength of light corresponding to the system. The activation will result in a cured system that incorporates the active component. Chemical reaction can be conducted by incorporating di-isocyanate, aldehyde, N-hydroxy -succinimide, di-imidazole, —NH2, —COOH, with a polymer such as PEG or HEMA. The process of photocuring and chemical reaction is known to one of ordinary skill in the art. U.S. Pat. No. 5,780,044, issued to Yewey et al. describes the formation of controlled release implants from liquid components.

In another embodiment, the active component is formulated in a liquid and delivered into a blood vessel through a drug delivery pump. The drug delivery pump may be adapted to be in fluid communication with an intravenous catheter implanted into a blood vessel, and the pump delivers the active component through the intravenous catheter into the blood vessel. The drug delivery pump may be implantable or non-implantable.

Some of the embodiments of the invention will be illustrated by the following set forth examples which are being given by way of illustration only, and not by way of limitation. All parameters are not to be construed to unduly limit the scope of the embodiments of the invention.

### EXAMPLE 1

1.5 grams of poly-(n-butyl methacrylate) and 0.5 gram of prednisolone can be dissolved in 100 ml of cyclohexanone and sprayed on a stent using standard small scale spray coating equipment like that available from EFD, Inc. East Providence, R.I. The stents can be dried at 75°C., under vacuum for 3 hours. Subsequently, they can be overcoated, using the same methods, with a solution of 0.6% benzalkonium heparin in AMS Techspray (Tech Spray Inc. Amarillo, Tex.), and dried for 10 minutes at 75°C. The resulting coated stents can have reduced thrombogenicity because of their heparin coating, and can release the anti-inflammatory drug prednisolone for several days.

### EXAMPLE 2

Same as Example 1, but prednisolone is replaced with dexamethasone.

### EXAMPLE 3

Same as Example 2, but benzalkonium heparin is replaced with tridodedecyl methylammonium heparin (TDMEC heparin).

### EXAMPLE 4

1.5 grams of poly-(n-butyl methacrylate) and 0.5 gram of prednisolone can be dissolved in 100 ml of cyclohexanone and sprayed on a stent using standard small scale spray coating equipment like that available from EFD, Inc. East Providence, R.I. The stents can be dried at 75°C., under vacuum for 3 hours. Subsequently, the stents can be overcoated with parylene, and the parylene is functionalized with amine groups by treatment with an ammonia plasma. The over coating and functionalization are standard industrial processes. The amine groups can then be reacted with partially oxidized heparin, binding the heparin to the surface of the parylene by Shiff's base formation, forming a thromboresistant heparin coating.

### EXAMPLE 5

1.5 grams of poly-(n-butyl methacrylate) and 0.5 gram of prednisolone and 0.5 gram of acetyl salicylic acid can be dissolved in 100 ml of cyclohexanone/methanol (50/50) and sprayed on a stent using standard small scale spray coating equipment like that available from EFD, Inc. East Providence, R.I. The stents can be dried at 75°C., under vacuum for 3 hours. The prednisolone can provide long term anti-inflammatory action, while aspirin can provide both short term anti-inflammatory action as well as thromboresistance due to its anti-platelet activity.

### EXAMPLE 6

Same as example 5, but acetyl salicylic acid can be replaced by clopidogrel.

### EXAMPLE 7

1.5 gram of poly-(n-butyl methacrylate) and 0.5 gram of prednisolone and 0.5 gram of benzalkonium heparin can be dissolved in 100 ml of cyclohexanone/Techspray (10/90) and sprayed on a stent using standard small scale spray coating equipment like that available from EFD, Inc. East Providence, R.I. The stents can be dried at 75°C., under vacuum for 3 hours.

### EXAMPLE 8

1.5 gram of poly-(n-butyl methacrylate) and 0.5 grams of rapamycin are dissolved in 100 ml of cyclohexanone/methanol (50/50) and can be sprayed on a stent using standard small scale spray coating equipment like that available from EFD, Inc. East Providence, R.I. The stents can be dried at 75°C., under vacuum for 3 hours. Subsequently, the stents can be overcoated, using the same methods, with a solution of 0.6% benzalkonium heparin in AMS Techspray (Tech Spray Inc. Amarillo, Tex.), and dried for 10 minutes at 75°C. The resulting coated stents can have reduced thrombogenicity because of their heparin coating, and can release rapamycin for several days. Rapamycin, in addition to being a potent immune suppressor, also has anti-inflammatory activity.

### EXAMPLE 9

1.5 grams of poly-(ethylene vinyl alcohol-co ethylene) (EVAL or EVOH) and 0.5 gram of prednisolone can be dissolved in 100 ml of dimethylsulfoxide (DMSO) and sprayed on a stent using standard small scale spray coating equipment like that available from EFD, Inc. East Providence, R.I. The stents can be dried at 75°C., under vacuum for 12 hours. Subsequently, the stents can be overcoated, using the same methods, with a solution of 0.6% benzalkonium heparin in AMS Techspray (Tech Spray Inc. Amarillo, Tex.), and dried for 10 minutes at 75°C. The resulting coated stents can have reduced thrombogenicity because of their heparin coating, and can release the anti-inflammatory drug prednisolone for several days.

### EXAMPLE 10

1.5 gram of poly-(n-butyl methacrylate) and 0.5 gram of prednisolone can be dissolved in 100 ml of cyclohexanone and sprayed on a stent using standard small scale spray coating equipment like that available from EFD, Inc. East Providence, R.I. The stents can be dried at 75°C., under vacuum for 3 hours. Subsequently, the system is overcoated with a thin layer of PTFE, using a commercially available method (such as that described by Advanced Surface Engineering, Inc, Eldersburg, Md.). The low surface energy of the teflon coating can prevent protein deposition, and subsequent thrombus accumulation, while the prednisolone can provide the anti-inflammatory component.

### EXAMPLE 11

### Reduction in Restenosis in the Porcine Coronary Artery Model

Porcine coronary models can be used to assess the synergism of the embodiments of the present invention. The degree of the inhibition of neointimal formation in the coronary arteries of a porcine stent injury model post stent therapy is predicted.

The preclinical animal testing should be performed in accordance with the NIH Guide for Care and Use of Laboratory Animals. Domestic swine can be utilized to evaluate the inhibition of the neointimal formation. Each testing procedure, excluding the angiographic analysis at the follow-up endpoints, should be conducted using sterile techniques. Base line blood samples should be collected for each animal before initiation of the procedure. Quantitative coronary angiographic analysis (QCA) and intravascular ultrasound (IVUS) analysis can be used for vessel size assessment.

The vessels at the sites of the delivery should be denuded by inflation of the PTCA balloons to 1:1.2 balloon to artery ratio. Stents, such as those described in Examples 1-10 are deployed at the delivery site such that final stent to artery ratio is, for example, 1.1:1.

QCA and IVUS analyses can be used for stent deployment guidance. Quantitative analysis of the stented coronary arteries to compare pre-stenting, post-stenting, follow-up minimal luminal diameters, stent recoil, and balloon/stent to artery ratio should be performed. Following stent implantation and final angiogram, all devices should be withdrawn and the wounds closed; the animals must be allowed to recover from anesthesia as managed by the attending veterinarian or animal care professionals at the research center.

Upon return to the research laboratory at, for example, the 28-day endpoint, angiographic assessments should be performed. Coronary artery blood flow is assessed and the stented vessels are evaluated to determine minimal lumen diameter. The animals are euthanized following this procedure at the endpoint. Following euthanasia, the hearts are pressure perfusion fixed with formalin and prepared for histological analysis, encompassing light microscopy, and morphometry. Morphometric analysis of the stented arteries includes assessment of the position of the stent struts and determination of vessel/lumen areas, percent (%) stenosis, injury scores, intimal and medial areas and intima/media ratios. Percent stenosis is quantitated by the following equation:

100 (IEL area−lumen area)/IEL area

where IEL is the internal elastic lamia.

It is believed that the percent restenosis in the treated groups will be significantly reduced.

While particular embodiments of the present invention have been shown and described, it will be clear to those of ordinary skill in the art that changes and modifications can be made without departing from this invention in its broader aspects and, therefore, the appended claims are to encompass within their scope all such changes and modifications as fall within the scope of this invention.

What is claimed is:

1. A method for inhibiting restenosis of a blood vessel, comprising the acts of:
a. providing a device carrying an active component, the active component comprises at least one anti-thrombotic substance and at least one anti-inflammatory substance; and
b. implanting the device into the blood vessel to inhibit restensosis of the blood vessel.

2. The method of claim 1, wherein the device is selected from a group of balloon-expandable stents, self-expandable stents, and grafts.

3. The method of claim 1, wherein
the anti-thrombotic substance is selected from a group of heparin, sodium heparin, low molecular weight heparin, hirudin, argatroban, forskolin, vapiprost, prostacyclin and prostacyclin analogs, D-phe-pro-arg-chloromethylketone, dipyridamole, glycoprotein IIb/IIIa platelet membrane receptor antibody, and recombinant hirudin; and
the anti-inflamatory substance is selected from a group of aspirin, diclofenac, etodolac, ibuprofen, ketoprofen, ketorolac, nabumetone, naproxen, oxaprozin, clobetasol, diflucortolone, flucinolone, halcinolonide, halobetasol, dexamethasone, betamethasone, corticol, cortisone, prednisone, and prednisolone.

4. The method of claim 1, wherein the device is coated with an ethylene vinyl alcohol copolymer and the active component is contained in the ethylene vinyl alcohol copolymer.

5. A stent comprising a generally tubular structure for implantation in a mammalian blood vessel, wherein the stent is coated with an anti-thrombogenic material which is not substantially released from the stent when the stent is implanted in the blood vessel and an anti-inflammatory substance contained in the coating and capable of being released from the coating when the stent is implanted.

6. The stent of claim 5, wherein the coating is made from a hydro-gel.

7. The stent of claim 5, wherein the coating is made from a hydro-gel selected from a group of poly-ethylene oxide, albumin, hydrophilic poly-methacrylates and hydrophilic poly urethanes.

8. A stent comprising pores formed in the surface wherein the sent is made from an anti-thrombogenic material and wherein the pores contain an anti-inflammatory substance.

9. The stent of claim 8, wherein the anti-inflammatory substance is selected from a group of aspirin, diclofenac, etodolac, ibuprofen, ketoprofen, ketorolac, nabumetone, naproxen, oxaprozin, clobetasol, diflucortolone, flucinolone, halcinolonide, halobetasol, dexamethasone, betamethasone, corticol, cortisone, prednisone, and prednisolone.

10. A stent for inhibiting restenosis of a mammalian blood vessel, comprising a generally tubular structure and carrying an active component, wherein the active components compirses an anti-thrombogenic substance and an anti-inflammatory substance.

11. The stent of claim 10, wherein
the anti-thrombotic substance is selected from a group of heparin, sodium heparin, low molecular weight heparin, hirudin, argatroban, forskolin, vapiprost, prostacyclin and prostacyclin analogs, D-phe-pro-arg-chloromethylketone, dipyridamole, glycoprotein IIb/IIIa platelet membrane receptor antibody, and recombinant hirudin; and
the anti-inflammatory substance is selected from a group of aspirin, diclofenac, etodolac, ibuprofen, ketoprofen, ketorolac, nabumetone, naproxen, oxaprozin, clobetasol, diflucortolone, flucinolone, halcinolonide, halobetasol, dexamethasone, betamethasone, corticol, cortisone, prednisone, and prednisolone.

12. The stent of claim 10, wherein the stent has an ethylene vinyl alcohol coating which contains the active component.

13. A polymeric matrix comprising an active component for inhibiting the migration or proliferation of smooth cells wherein the active component inhibits the formation of thrombus and inhibits the infiltration of inflammatory cells in the thrombus.

14. The polymeric matrix of claim 13, wherein the polymer is a liposome.

15. The polymeric matrix of claim 13, wherein the polymer is an ethylene vinyl alcohol copolymer.
